(19) United States
(12) Patent Application Publication
(10) Pub. No.: US 2023/0111334 A1
(43) Pub. Date: Apr. 13, 2023

(54) SYSTEMS FOR TREATING PULMONARY ARTERIAL HYPERTENSION THROUGH NEUROMODULATION

(71) Applicant: ARIZONA BOARD OF REGENTS ON BEHALF OF THE UNIVERSITY OF ARIZONA, Tucson, AZ (US)

(72) Inventors: Mohamed Ahmed, Tucson, AZ (US); Philipp Gutruf, Tucson, AZ (US)

(21) Appl. No.: 17/794,558

(22) PCT Filed: Jan. 27, 2021
(86) PCT No.: PCT/US2021/015259

§ 371 Date: Jul. 21, 2022

Related U.S. Application Data
(60) Provisional application No. 62/967,373, filed on Jan. 29, 2020.

(51) Int. Cl. A61N 1/36; A61N 1/378

(52) U.S. Cl. CPC A61N 1/36117; A61N 1/3787; A61N 1/36031; A61N 1/36139

(57) ABSTRACT

Systems and methods for treating pulmonary arterial hypertension (PAH) featuring a device that targets the vagal nerve fiber looped around both right and left main bronchi. The system comprises digital and analog electronics; a strain sensor for measuring arterial pressure operatively connected to the electronics, and a stimulator for selectively stimulating a vagus nerve operatively connected to electronics. The system is a closed- loop system. The strain sensor has sufficient flexibility to wrap around an artery. The system stimulates the vagus nerve with specific electrical signals that relax the smooth muscle of pulmonary vascular tree. The electric stimulation can be controlled to achieve localized pulmonary vascular smooth muscle relaxation with non or minimal systemic side effects.

### CROSS-REFERENCES TO RELATED APPLICATIONS

This application claims benefit of U.S. Provisional Application No. 62/967,373 filed Jan. 29, 2020, the specification of which is incorporated herein in its entirety by reference.

### BACKGROUND OF THE INVENTION

### Field of the Invention

The present invention relates to treatments for pulmonary arterial hypertension (PAH), more particularly to a system for treating PAH that features electrical stimulation of the vagus nerve. The system is a closed-loop system and features specific vagus nerve stimulation, pulmonary arterial pressure acquisition, and decision rule evaluation, which together guides closed-loop regulation of pulmonary arterial pressure.

### Background Art

Pulmonary arterial hypertension (PAH) is a potentially fatal disease that leads to remodeling of blood vessels in the lung, progressively low oxygen in the blood, right sided heart failure and death. PAH affects all ages, races and ethnic backgrounds. In neonates, birth depression, underdeveloped lungs or congenital heart disease are amongst the risk factors. In adults, PAH often occurs in association with chronic lung disorders, sickle cell anemia, scleroderma, lupus, or sleep apnea, with hypoxia playing a pivotal role in the etiology.

Among neonatal patients, there are limited therapies that have been approved for the treatment of PAH. Currently, nitric oxide is one existing treatment, but it is not widely used. A number of drugs have been recently developed to treat one type of pulmonary hypertension directly, but other types are often approached in terms of treating the underlying disease or even surgically.

### BRIEF SUMMARY OF THE INVENTION

The present invention features systems and methods for treating pulmonary arterial hypertension (PAH) using electrical stimulation of the vagus nerve. The system is a closed-loop system and features specific vagus nerve stimulation, pulmonary arterial pressure acquisition, and decision rule evaluation, which together guides closed-loop regulation of pulmonary arterial pressure. The closed-loop systems herein may feature a continuous, repeated cycle of actuation-observation-update. For example, the closed-loop system may incorporate a cycle of nerve stimulation-blood pressure acquisition-update, wherein the update is related to a decision rule implemented in an electronic circuit (e.g., microprocessor) that determines the next nerve stimulation (if any) for achieving a goal of particular pulmonary arterial pressure.

Without wishing to limit the present invention to any theory or mechanism, it is believed that the methods and systems of the present invention are advantageous because they have few to no side effects, especially compared to drug treatments, the technique can be applied to all groups of PAH. The system does not require frequent repeated visits to a healthcare provider as compared to certain drugs that require repeated infusions.

The present invention features a closed-loop system for treating or ameliorating symptoms of pulmonary arterial hypertension (PAH). In certain embodiments, the system comprises (a) a mechanism for observing a state of a set of variables, the set of variables including one or a combination of: pulmonary arterial pressure, right ventricular pressure, systemic blood pressure, and heart rate; (b) a mechanism for deciding upon an appropriate stimulus to apply to a vagus nerve in order to achieve a target state based on the observed state of the set of variables in (a); and (c) a mechanism for applying the appropriate stimulus to the vagus nerve decided upon in (b) in order to achieve the target state. The system continuously activates mechanisms (a), (b), and (c) in order in a continuous loop, wherein the target state is a physiological goal for treating or ameliorating symptoms of PAH.

In some embodiments, the target state is a target pulmonary arterial pressure. In some embodiments, the mechanism for observing a pulmonary arterial pressure is a strain sensor. In some embodiments, the strain sensor is wrapped around a pulmonary artery. In some embodiments, the mechanism for applying the appropriate stimulus to the vagus nerve is an electrical stimulator. In some embodiments, the mechanism for applying the appropriate stimulus to the vagus nerve targets a vagal nerve fiber looped around both right and left main bronchi.

In some embodiments, the system is implantable. In some embodiments, the system is not implantable. In some embodiments, the system is introduced via esophageal access. In some embodiments, the system is powered via a wireless mechanism. In some embodiments, the system records arterial pressure. In some embodiments, the system records arterial pressure non-invasively.

The present invention also features a closed-loop system for treating or ameliorating symptoms of pulmonary arterial hypertension (PAH). In some embodiments, the system comprises electronics; a strain sensor for measuring arterial pressure, the strain sensor has sufficient flexibility to wrap around an artery and is operatively connected to the electronics; and a stimulator for selectively stimulating a vagus nerve, the stimulator is operatively connected to the electronics. The system is a closed-loop control system that regulates a patient’s arterial pressure to treat or ameliorate symptoms of PAH.

In some embodiments, the electronics are analog electronics, a microprocessor, an application-specific circuit, a field programmable array, or a combination thereof.

In some embodiments, the system is implantable. In some embodiments, the system is not implantable. In some embodiments, the system is introduced via esophageal access.

In some embodiments, the strain sensor detects arterial pressure and sends said arterial pressure to the electronics, whereupon the electronics determines whether or not the arterial pressure is such that the stimulator should be activated; wherein when the arterial pressure is such that the stimulator should be activated, the electronics sends a signal to the stimulator to stimulate the vagus nerve.

In some embodiments, the stimulator targets the vagal nerve fiber looped around both right and left main bronchi. In some embodiments, the system relaxes the smooth muscle of the pulmonary vascular tree. In some embodiments, the system is powered via a wireless mechanism. In some embodiments, the system records arterial pressure. In some embodiments, the system records arterial pressure non-invasively.

The present invention also features a method of treating pulmonary arterial hypertension (PAH) in a patient in need thereof. In some embodiments, the method comprises using a system according to the present invention, wherein the system is effective for selective stimulation of a vagus nerve to relieve symptoms of or treat PAH.

One of the unique and inventive technical features of the present invention is the implementation of a microprocessor capable of executing a machine learning algorithm in a seamlessly implanted device continuously powered via a wireless, battery-free mechanism. Without wishing to limit the invention to any theory or mechanism, it is believed that the technical feature of the present invention advantageously provides for a non-intrusive and compact vagal nerve stimulation device that is capable of long-term, accurate, and continuous monitoring and treatment of a PAH patient while obviating the need for recharging the device. None of the presently known prior references or work has the unique inventive technical feature of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows a photograph of the device of the present invention comprising a loop antenna, off-the-shelf electronic components, serpentine interconnects, a soft pressure sensor, and soft vagal nerve stimulation electrodes.

FIG. 1B shows a close-up of the vagal nerve stimulation electrodes.

FIG. 1C shows a close-up view of the soft pressure sensor.

FIG. 1D shows a general schematic view of an embodiment of the system of the present invention. In certain embodiments, the serpentine interconnection can stretch. U.S. Pat. No. 10,840,536 B2“Stretchable Electronic Systems with Containment Chambers” by John A. Rogers implements this well-known technology. Note, in certain embodiments, a first serpentine interconnect extends from the electronic components to the strain sensor and a second extends from the electronic components to the stimulation electrodes. Note, the two cords may not necessarily be co-located.

FIG. 2 shows a block diagram of certain functional blocks in an embodiment of the system of the present invention.

FIG. 3 shows a schematic view of the components of certain embodiments of the system of the present invention.

FIG. 4 shows a schematic view of the system of the present invention wherein arterial pressure is measured via the pressure sensor, the system calculates an appropriate stimulation based on the arterial pressure (also based on the individual), and executes a stimulation.

FIG. 5 shows right ventricular pressure (RVP) before, during, and after vagal nerve stimulation (VNS) (using intensity of 1500 uA, frequency of 30 Hz, for duration of 20 uS, for a total of 30 seconds).

FIG. 6 shows beat to beat analysis, showing that specific target for certain vagal nerve fibers can induce relaxation of contracted pulmonary vascular tree and significant drop of right ventricular pressure (RVP) without any significant or marked change in systemic pressure, heart rate or breathing pattern (no apnea).

FIG. 7 shows ECHO findings in rat models with pulmonary arterial hypertension (PAH) induced by hypoxia and Sugen after prolonged vagal nerve stimulation (VNS).

FIG. 8 shows ex-vivo studies of extracted pulmonary artery (PA) relaxation response to NO in both stimulated vs. non-stimulated rat models with pulmonary arterial hypertension (PAH).

FIG. 9A shows a graph of the RF power harvesting capability, i.e. rectified voltage and harvested power as functions of electrical load, of the loop antenna of the present invention.

FIG. 9B shows a 3D graph of the spatial distribution of harvested power in a rat home cage. This is an example of power availability, as the antenna of the present invention can be altered to power the device inside a patient’s body.

FIG. 10A shows a graph of a change in resistance of the soft pressure sensor of the present invention as a function of pressure.

FIG. 10B shows a graph of the change in resistance of the soft pressure sensor of the present invention upon progressively varying pressure.

FIG. 11A shows current and voltage profiles of a biphasic stimulation pulse with progressively increasing stimulation current in the present invention.

FIG. 11B shows current profiles of biphasic pulses under driving voltage of 18 V and -18 V, with increasing load in the present invention.

### DETAILED DESCRIPTION OF THE INVENTION

The present invention features systems and methods for treating pulmonary arterial hypertension (PAH) using bilateral electrical stimulation of the vagus nerve. The system is a closed-loop system and features specific vagus nerve stimulation, pulmonary arterial pressure acquisition, and decision rule evaluation, which together guides closed-loop regulation of pulmonary arterial pressure.

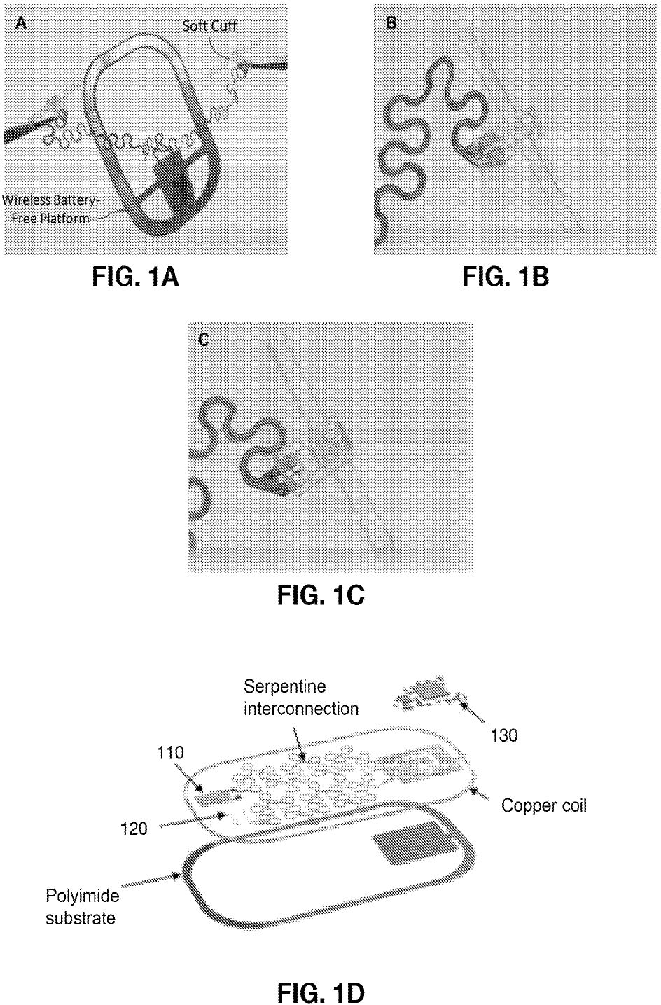

For example, the present invention features a device that targets the vagal nerve fiber looped around both right and left main bronchi. Referring to FIG. 1D, the system comprises a sensor, e.g., a strain sensor operating as a pulmonary arterial pressure-sensing cuff (110), and an electrical stimulator exciting the vagus nerve, e.g., stimulation electrodes (120). The sensor and stimulator are operatively connected to electronics (130) that are wirelessly powered. The electronics (130) may be powered via battery. In some embodiments, the electronics (130) are wirelessly powered (e.g., wireless electronic stimulator). The present invention is not limited to the aforementioned electronics or power systems.

The system is regulated in a closed loop, e.g., the system combines observation and stimulation in a closed-loop mechanism. As used herein, a closed loop may generally refer to a system that observes all of the state variables of interest in the system (e.g., pulmonary arterial pressure, right ventricular pressure, etc., optionally other hidden’ state variables such as but not limited to systemic blood pressure, heart rate, and breathing rate, etc.) that help measure the patient’s well-being, which may be observed as right ventricular pressure (RVP) or correlated with right ventricular pressure (RVP). The system decides upon a suitable stimulus to apply to the system in order to guide it towards its target state or goal state, then applies that stimulus to the system, and so on, in a continuous loop.

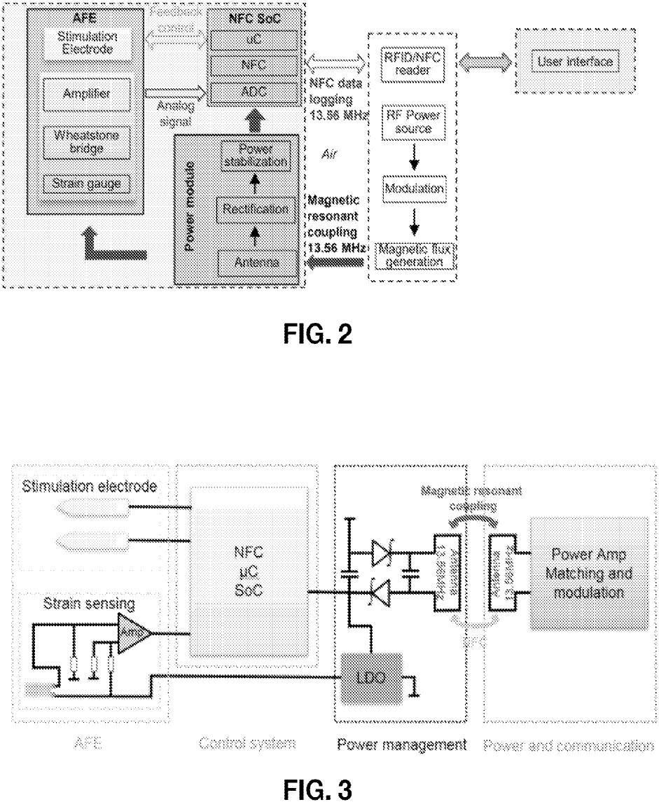

FIG. 2 and FIG. 3 show schematic views of the components of the system of the present invention. The present invention is not limited to the materials and configurations shown herein.

In some embodiments, the electronics (130) may comprise a microprocessor capable of executing computer-executable instructions and a memory component comprising computer-executable instructions to act as a mechanism for deciding upon an appropriate stimulus to apply to a vagus nerve in order to achieve a target state based on the observed state of the set of variables observed by the sensor (110). The computer-executable instructions may comprise a model created by an unsupervised machine learning algorithm, a supervised machine learning algorithm, or reinforcement learning. An unsupervised machine learning algorithm is defined as an algorithm where an Al is given a training set and develops its own classification based on said training set, i.e. identifiable differences between an unhealthy reading and a healthy reading from the sensor (110). A supervised machine learning algorithm is defined as an algorithm where an Al is given both a training set and a set of features for the Al to identify and use in classification/decision-making. The training set may comprise a plurality of goal state sensor readings and a plurality of sensor readings that would require vagal nerve stimulation. Reinforcement learning is defined as a method of training an Al that involves allowing it to make choices in how to classify data and either rewarding or penalizing the Al to reinforce the correct decisions. Once the model has been generated and validated through one or more of the above methods, it is deployed to the memory component of the electronics (130). The electronics (130) of the present invention may be capable of taking inputs and deciding which output is appropriate based on the model (a process known as “inference”).

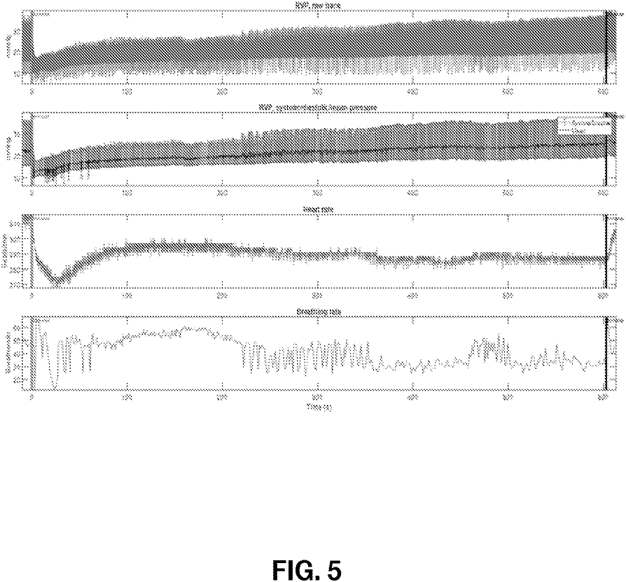

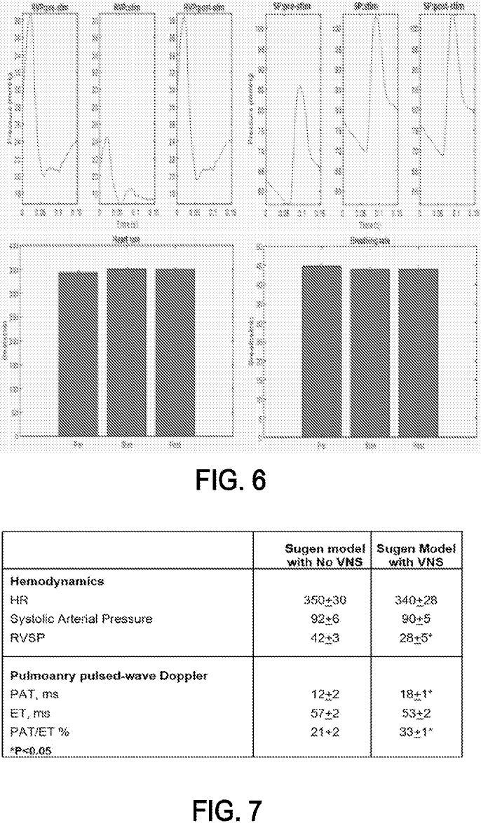

The system is designed to be flexible and stretchable. When the system is implanted, the strain sensor is wrapped (cuffed) around the artery to measure arterial pressure. The stimulator is configured to stimulate part of the vagus nerve in a specific manner. The system stimulates the vagus nerve with specific electrical signals that relax the smooth muscle of pulmonary vascular tree. The electric stimulation can be controlled to achieve localized pulmonary vascular smooth muscle relaxation with non or minimal systemic side effects. For example, without wishing to limit the present invention to any theory or mechanism, the system of the present invention is designed to have no (or minimal) impact on heart rate, systemic pressure, and breathing rate. FIG. 5 shows an example of how the system can have minimal impact on heart rate, systemic pressure, and breathing rate.

For humans, the approach may use natural access though a probe, e.g., esophageal access, or an implant under the skin, muscle, bone or cartilage, through which one can access the vagal nerve fibers.

The system may be implanted such that the sensor is cuffed around the artery. The system measures arterial pressure via the sensor and evaluates the measured arterial pressure via an electronic circuit (e.g., microprocessor, etc.). The electronic circuit, upon determining that stimulation is required, sends a signal to the stimulator to provide stimulation to the vagus nerve in an appropriate manner.

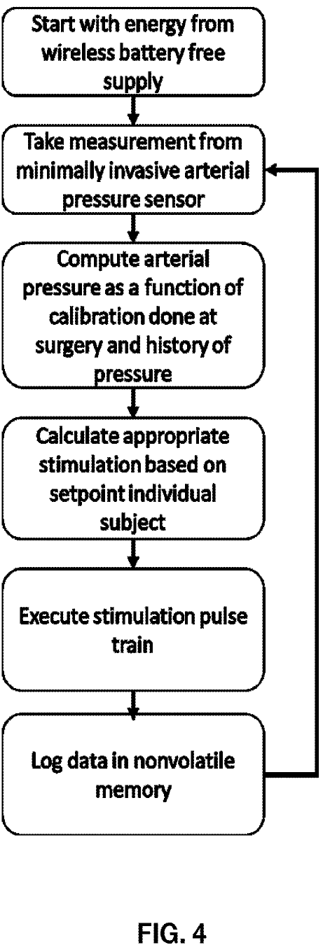

FIG. 4 shows a schematic view of how the system of the present invention functions. For example, arterial pressure is measured via the pressure sensor wrapped around the artery. The system computes arterial pressure as a function of calibration done at surgery and history of the pressure, then calculates an appropriate stimulation. The system then executes a stimulation.

### EXAMPLE

The following is a non-limiting example of the present invention. It is to be understood that said example is not intended to limit the present invention in any way. Equivalents or substitutes are within the scope of the present invention.

Example 1 describes an approach to treating PAH, wherein vagal nerve stimulation (VNS) is used to directly stimulate pulmonary vagal nerve fibers that supply both bronchial and pulmonary vascular tree only (no innervation to the heart). Without wishing to limit the present invention to any theory or mechanism, it is believed that the approach can decrease and attenuate the PAH severity, which could be followed by oxygenation improvement to allow healing and to provide a healthy environment. Also, the approach can be easily applied in different settings, e.g., ICU setting, at primary care, etc.

Murine model with PAH induced by chroncuhypoxia, and mitigated by VNS: animal model of PAH induced by hypoxia exposure for 3 weeks (FiO₂ 10%), was sedated and monitored for both right ventricular pressure (RVP), and systemic pressure. Continuous monitoring of the vital signs including: heart rate (HR), EKG, blood oxygen saturation, breathing rate and both pulmonary and systemic pressures; were recorded using a computerized hemodynamic recording system (HAEMODYN, Harvard Apparatus, MA, USA), Pulmonary and systemic pressures were measured through cannulation of right internal jugular vein and femoral artery. Cervical vagal nerve was exposed and a cuffed sling bipolar platinum electrode (Plastics One), was placed underneath the nerve trunk. Continuous monitoring/stimulation of vagal nerve activity was established using a stimulation module (MCS STG4008) controlled by the Acknowledge software (Biopac Systems).

Stimulation was delivered using different matching stimulation parameters (current intensity Amplitude, pulse width, pulsing frequency, and pulsing train. Different patterns were used in a trial to optimize the stimulation of specific nerve fibers, which supply the vascular pulmonary smooth muscle with minimum side effect on other monitored vital signs, e.g., oxygen desaturation, apnea, bradycardia, arrhythmia and systemic hypotension. Using this specific electric stimulation with specific parameters (intensity, frequency and duration), inventors were able to target specific nerve fiber to induce smooth muscle relaxation of the pulmonary vascular bed, which lead to instant drop of RVP; during the stimulation; to its normative values. (See FIG. 5)

Analysis of the data using beat to beat analysis (see FIG. 4) showed interestingly that specific target for certain vagal nerve fibers can induce relaxation of contracted pulmonary vascular tree and significant drop of RVP, without any significant or marked change in systemic pressure, heart rate or breathing pattern (no apnea). Similar data was obtained using different murine animal models with a wide range of PH severity and with variable degree of pulmonary vascular remodeling including rats with PH induced by hypoxia and Sugen (a vascular endothelial growth factor receptor antagonist). A specific pattern of VNS (with a safe margin) was able to be defined; the pattern of VNS can trigger the vascular smooth muscle relation with none or minimal systemic or unwanted side effects (including hypotension, apnea and bradycardia).

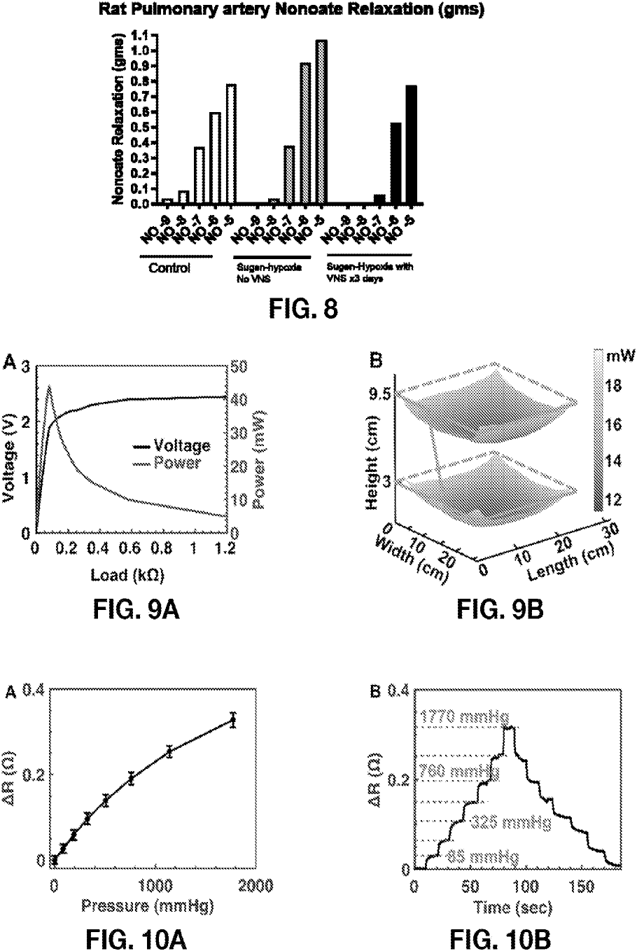

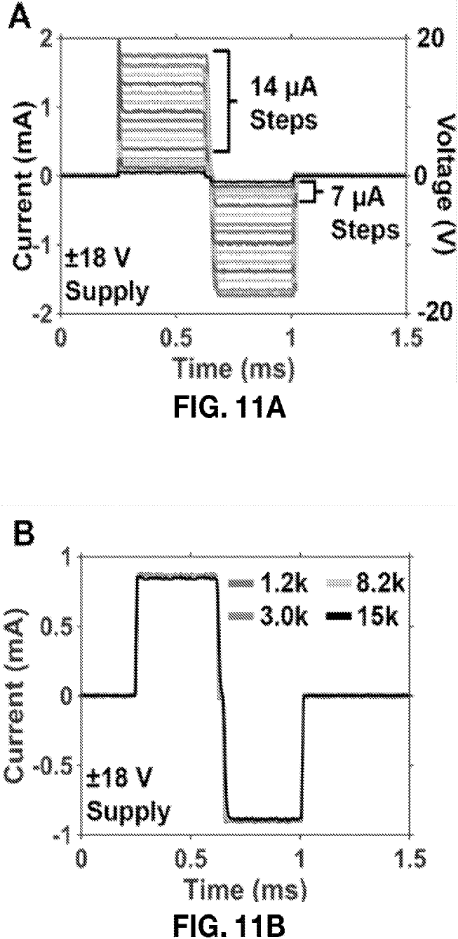

Using an adult rat with chronic progressive PAH induced by hypoxia (FiO₂ 10% for 3 weeks) and Sugen (sigma) (3 doses), prolonged intermittent VNS for 5-7 days, showed a significant improvement in cardiac function; mainly reduction of right ventricular systolic pressure (RVSP), increase pulmonary artery acceleration time (PAT) and significant increase of PATIET% (Right ventricle ejection time) (see FIG. 7). Extracted medium sized pulmonary artery (PA) from a stimulated group, showed histopathological significant remodeling and a significant reactive to NO in ex-vivo setting compared to a non-stimulated group (see FIG. 8).

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

1. A closed-loop system for treating or ameliorating symptoms of pulmonary arterial hypertension (PAH), the system comprises:
a. a mechanism for observing a state of a set of variables, the set of variables including one or a combination of: pulmonary arterial pressure, right ventricular pressure, systemic blood pressure, and heart rate;
b. a mechanism for deciding upon an appropriate stimulus to apply to a vagus nerve in order to achieve a target state based on the observed state of the set of variables in (a); and
c. a mechanism for applying the appropriate stimulus to the vagus nerve decided upon in (b) in order to achieve the target state;
wherein the system continuously activates mechanisms (a), (b), and (c) in order in a continuous loop, wherein the target state is a physiological goal for treating or ameliorating symptoms of PAH.

2. The system of claim 1, wherein the target state is a target pulmonary arterial pressure.

3. The system of claim 1, wherein the mechanism for observing a pulmonary arterial pressure is a strain sensor.

4. The system of claim 3, wherein the strain sensor is wrapped around a pulmonary artery.

5. The system of claim 1, wherein the mechanism for applying the appropriate stimulus to the vagus nerve is an electrical stimulator.

6. The system of claim 1, the mechanism for applying the appropriate stimulus to the vagus nerve targets a vagal nerve fiber looped around both right and left main bronchi.

7. The system of claim 1, wherein the system is implantable.

8. The system of claim 1, wherein the system is not implantable.

9. The system of claim 8, wherein the system is introduced via esophageal access.

10. The system of claim 1, wherein the system is powered via a wireless mechanism.

11. The system of claim 1, wherein the system records arterial pressure.

12. The system of claim 1, wherein the system records arterial pressure non-invasively.

13. The system of claim 1, wherein the mechanism for deciding upon an appropriate stimulus to apply to a vagus nerve implements one or more of supervised machine learning, unsupervised machine learning, and reinforcement learning.

14. A closed-loop system for treating or ameliorating symptoms of pulmonary arterial hypertension (PAH), said system comprising:
a. electronics;
b. a strain sensor for measuring arterial pressure, the strain sensor has sufficient flexibility to wrap around an artery and is operatively connected to the electronics; and
c. a stimulator for selectively stimulating a vagus nerve, the stimulator is operatively connected to the electronics;
wherein the system is a closed-loop control system that regulates a patient’s arterial pressure to treat or ameliorate symptoms of PAH.

15. The system of claim 14, wherein the electronics are analog electronics, a microprocessor, an application-specific circuit. a field programmable array, or a combination thereof.

16. The system of claim 14, wherein the system is implantable.

17. The system of claim 14, wherein the system is not implantable.

18. The system of claim 17, wherein the system is introduced via esophageal access.

19. The system of claim 14, wherein the strain sensor detects arterial pressure and sends said arterial pressure to the electronics, whereupon the electronics determines whether or not the arterial pressure is such that the stimulator should be activated; wherein when the arterial pressure is such that the stimulator should be activated, the electronics sends a signal to the stimulator to stimulate the vagus nerve.

20. The system of claim 19, wherein the electronics comprise a microprocessor capable of executing computer-executable instructions and a memory component comprising computer-executable instructions.

21-27. (canceled)
